(19) United States
(12) Patent Application Publication
(10) Pub. No.: US 2011/0106119 A1
(43) Pub. Date: May 5, 2011

(54) ANASTOMOSIS DEVICE

(75) Inventors: Chih-Hsun LIN, Taipei City (TW); Ruey-Yug TSAY, Taipei City (TW)

(73) Assignee: NATIONAL YANG-MING UNIVERSITY, TAIPEI CITY (TW)

(21) Appl. No.: 12/698,477

(22) Filed: Feb. 2, 2010

(30) Foreign Application Priority Data
Oct. 30, 2009 (TW) 098136955

(51) Int. Cl. A61B 17/11

(52) U.S. Cl. 606/153

(57) ABSTRACT

The present invention discloses an microvascular anastomosis device. The device comprises two rotatable and movable ring bodies disposed relatively on a guiding base, and a guiding member lead two vessels to pass through the two ring bodies respectively, wherein a tenon structure is disposed at the two ring bodies for aligning and embedding each other, and inner surface of each vessel is pressured in axial symmetry to make the outer surface of each vessel adhered and fixed onto the inner surface of each ring body, so as not to turn the vessels inside out when performing microvascular Anastomsis.

### BACKGROUND OF THE INVENTION

1. Field of the Invention

The present invention generally relates to an anastomosis device, and more particularly to an microvascular anastomosis device without turning the vessel wall inside out when operating.

2. Description of the Related Art

The anastomosis of blood vessel is one of the most important achievement in contemporary surgery which expands the surgical field to help patients getting better treatment results. Operations such as Heart Bypass Surgery, Solid-Organ Transplantation, Radiocephalic Fistula, Microvascular Free Flap Reconstruction, and etc. are all required to be finished up with anastomosis of blood vessels. As for Microvascular Free Flap Reconstruction, the anastomosis of blood vessel makes it possible for the patients with congenital anomalies, malignant tumor invasion, or injured-caused massive soft tissue defects to be able to receive autograft to resurface the defects, and then improve function and appearance.

The present procedure of microvascular anastomosis is mainly hand-stitched.

However, the outcome of this traditional method for anastomosis of blood vessel relies on the skill and experience of the surgeon. It therefore may result in problems of taking long time, blood leakage from pinhole, or vessel occlusion due to the stitching of opposite side wall or twisting the blood vessels. To solve these problems, there are therefore studies for various anastomosis techniques.

In general, there are two types of sutureless anastomosis techniques, one by chemical and the other one by physical bonding, for tissue fixation. For the one by chemical bonding, it has been disclosed in the literatures to apply tissue glue or laser welding for the anastomosis of blood vessel. However, none of these two methods has any actual clinical applications developed due to their complicated and inconvenient operating procedure and the difficulty in maintaining a high patency.

Current clinical implementations of microvascular anastomosis techniques are mainly mechanical bonding. Based on their fixation mechanism, these mechanical tissue fixation methods include: stapling, clamping, coupling by a ring-shaped anastomotic coupler. For stapling, a launcher shoots an anastomotic staple to pierce the two ends of vessel walls intended to be anastomosed, and then bends the staple to fix the tissue. For clamping, a vascular clip is applied to hold the two ends of vessel walls to be anastomosed in a non-piercing way. However, because it is difficult to maintain the stress distribution in vessel walls uniformly, the vessel wall may be damaged easily by this method. To improve this, some ring-shaped couplers are developed in succession.

The fixation mechanisms of a ring-shaped anastomotic coupler are similar to those of stapling and clamping methods. Among them, the “ring-pin” type coupler is a design using stapling as the fixation mechanism and the “SYNOVIS” GEM microvascular anastomotic coupler system (SYNOVIS MICRO COMPANIES ALLIANCE, INC. USA) is the only ring-pin type coupler currently on the market. The “extraluminal cuffing ring” is a coupler using clamping as tissue holding mechanism, which does not have any commercialized products yet.

Compared to the traditional hand-stitched method, the mechanical bonding methods mentioned above indeed can effectively shorten the operation time and reduce the requirements in surgical skills of anastomosis. However, a common drawback of these methods was that the vessels have to be firstly everted for 90 degrees or even 180 degrees in operation, which is not applicable to the vessel wall with atherosclerotic change and may cause vessel spasm due to tension at the anastomosis site, the consequence of insufficient vessel length for eversion. This drawback had substantially restricted the implementation of these methods in microvascular anastomosis. Also, these ring-shaped anastomotic couplers required a complicated staple launcher or alignment equipment which makes the system expensive. Furthermore, the anastomotic staple or ring-pin type anastomotic coupler fixed the tissue by piercing vessel walls, which inevitably damaged the vessel wall. The adherence by tissue clips or extraluminal cuffing ring also may cause pressure necrosis of vessel walls. Because of the distinct material properties of anastomotic couplers and vessel walls, it is possible to cause local compliance mismatch of the vessel wall, and subsequently disturbs the transmission of pulse waves in blood circulation.

In view of the limitation of recent anastomotic devices, the present invention modifies the prior piercing or clipping methods, and designs an anastomosis device needn't to evert the vessels in microvascular anastomosis, which is one of the serious problems in prior art.

### SUMMARY OF THE INVENTION

An objective of the present invention is to provide anastomosis devices capable of reducing the vessel damage during microvascular anastomosis, shortening the operation time, providing exterior support, and avoiding collapse of vessel wall so as to improve vessel patency while easy to be aligned and operated.

To achieve the objectives above, the present invention provides an anastomosis device comprising a guiding base, two ring bodies which are rotatable and movable on the guiding base for connecting two vessels, and a guiding member leading the two vessels to pass through the two ring bodies respectively.

Preferably, the two ring bodies have a tenon structure for aligning and embedding the two ring bodies.

Preferably, the inner surface of each vessel is pressured uniformly in axial symmetry to make the outer surface of each vessel adhered and fixed onto the inner surface of each ring body, and the outer surface of each vessel is adhered and fixed on the inner surface of each ring body by a way selected from the following methods: chemical bonding, physical adsorption, and mechanical retention, e.g. using a bio-glue or surface micro-needles.

Preferably, the two ring bodies have at least one thread hole/groove for providing a stitch to pass through and then linking the two vessels.

Preferably, the two ring bodies have a press-buckled structure for fixing the two vessels.

Preferably, the length of each ring body is between 0.5-10 mm, and the inner diameter of each ring body is between 0.5-10 mm.

A best illustrative embodiment of the invention with drawings is described as below.

### BRIEF DESCRIPTION OF THE DRAWINGS

All the objects, advantages, and novel features of the invention will become more apparent from the following detailed descriptions when taken in conjunction with the accompanying drawings.

FIG. 1 shows an assembly drawing of the anastomosis device in accordance with the invention.

FIG. 2A shows a side view of the anastomosis device in accordance with the invention before linking two vessels.

FIG. 2B shows a side view of the anastomosis device in accordance with the invention while linking two vessels.

FIG. 3 shows a side view of sectional drawing in accordance with the invention for leading vessels to ring bodies.

FIG. 4 shows a schematic diagram in accordance with the invention for applying uniform pressure on the inner surface of vessels in axial symmetry.

FIG. 5 shows a schematic diagram of a vessel connected with the ring bodies in accordance with the invention.

FIG. 6 shows an assembly drawing of one embodiment of the way of vessel anastomosis is in accordance with the invention.

FIG. 7 shows an assembly drawing of another embodiment of the way of vessel anastomosis in accordance with the invention.

### DETAILED DESCRIPTION OF THE PREFERRED EMBODIMENT

Although the invention has been explained in relation to several preferred embodiments, the accompanying drawings and the following detailed description are the preferred embodiment of the present invention. It is to be understood that the following disclosed descriptions will be examples of present invention, and will not limit the present invention into the drawings and the special embodiment.

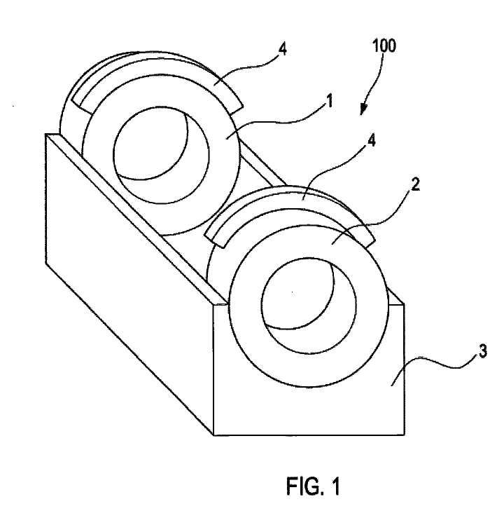

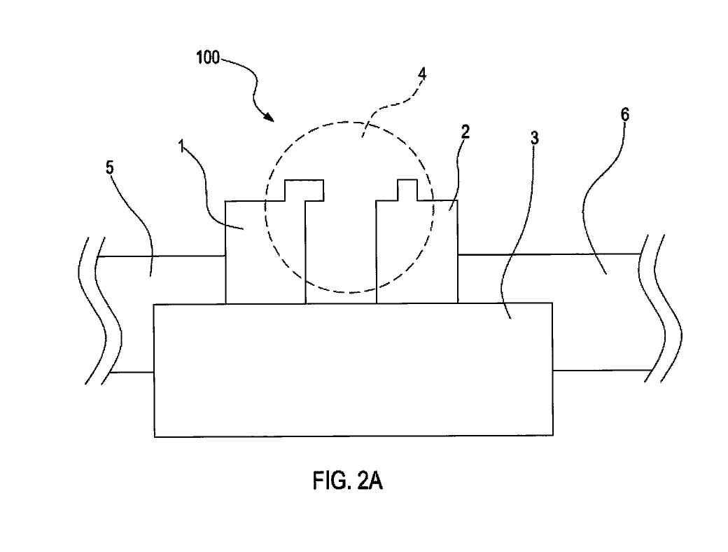

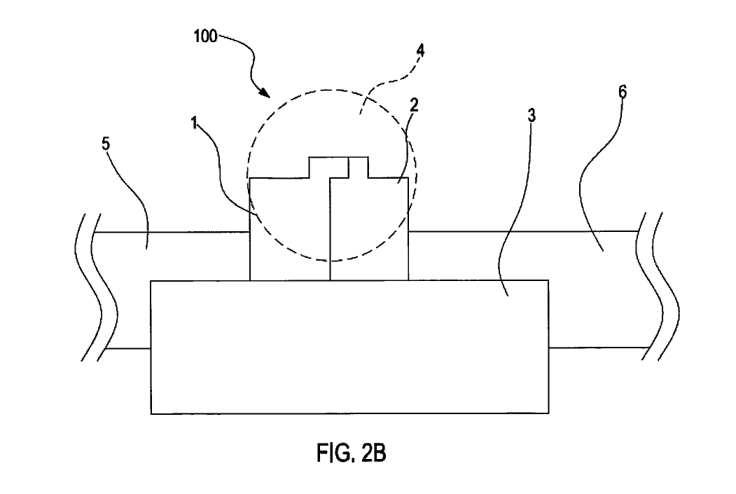

Please refer to FIG. 1, 2A and 2B, wherein FIG. 1 shows an assembly drawing of the anastomosis device in accordance with the invention, FIG. 2A shows a side view of the anastomosis device in accordance with the invention before linking two vessels, and FIG. 2B shows a side view of the anastomosis device in accordance with the invention while linking two vessels.

The anastomosis device 100 comprises a pair of ring bodies 1, 2 and a guiding base 3, wherein the ring bodies 1, 2 are two separated cylindrical ring-shaped members and have a tenon structure 4 with concave and convex portions capable of embedding each other, but not limit thereto. When anastomosing, the pair of ring bodies 1, 2 is disposed on the guiding base 3 and embedded each other by the tenon structure 4 after aligning so as to link two vessels 5, 6. The detail anastomosis operation will be described later.

The length and inner diameter of two ring bodies 1, 2 can be adjusted based on different vessels 5, 6, for example, the length is between 0.5-10 mm and the inner diameter is between 0.5-10 mm. Preferably, the length is between 1-5 mm and the inner diameter is between 1-6 mm, but not limit thereto.

The tenon structure 4 disposed at the ring bodies 1, 2 is aligned with concave and convex portions coaxially so as to link two vessels 5, 6 connected with the ring bodies 1, 2 respectively. A radius of curvature of a bearing of the guiding base 3 is the same as an outer diameter of each ring body 1 and 2 so as to limit the ring bodies 1, 2 to only shift forward and backward on the guiding base 3. And preferably, a length of the guiding base 3 is longer than the sum of the lengths the two ring bodies 1 and 2.

When aligning, the two ring bodies 1 and 2 connected with two vessels 5, 6 respectively are disposed on the guiding base 3 and rotated to adjust the linking direction to prevent the two vessels 5, 6 from twisting while linking. After confirming the linking direction, push the two ring bodies 1, 2 close to each other till the concave and convex portions of the tenon structure 4 contacts, and then finish the axial alignment.

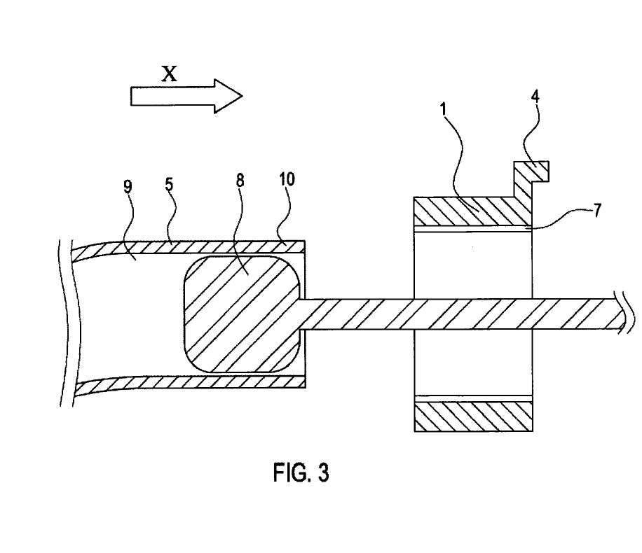

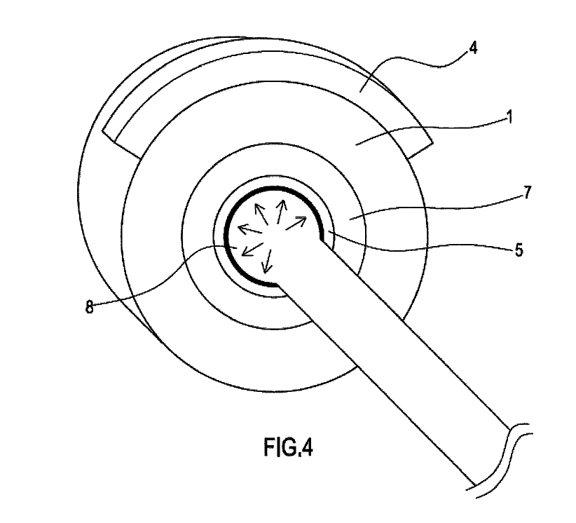

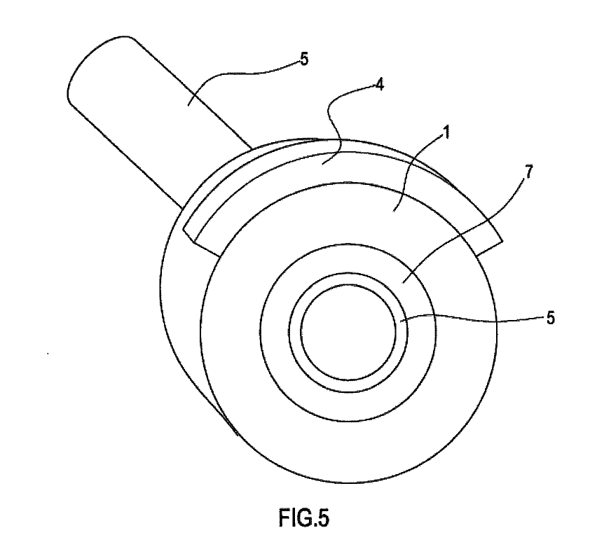

Please refer to FIG. 3, FIG. 4 and FIG. 5, wherein FIG. 3 shows a side view of sectional drawing in accordance with the invention for leading vessels to ring bodies, FIG. 4 shows a schematic diagram in accordance with the invention for applying uniform pressure on the inner surface of vessels in axial symmetry, and FIG. 5 shows a schematic diagram in accordance with the invention for connecting vessels with ring bodies.

This embodiment is described by ring body 1, because the action of ring body 2 is the same as ring boy 1. When tending to link vessel 5, firstly, the vessel 5 is passed through the ring body 1 by the guiding member 8, shown as direction X of FIG. 3, and then an inner surface 9 of vessel 5 is pressured uniformly in axial symmetry to make the outer surface 10 of vessel 5 adhere and fix on an inner surface 7 of ring body 1, wherein the outer surface 10 of vessel 5 is adhered and fixed on the inner surface 7 of ring body 1 by one of chemical bond, physical adsorption (ex. coating bio-gel) and mechanical retention (ex. distributing fine needles).

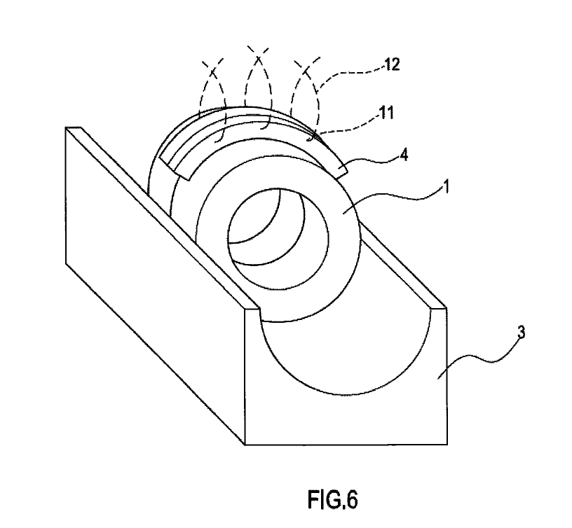

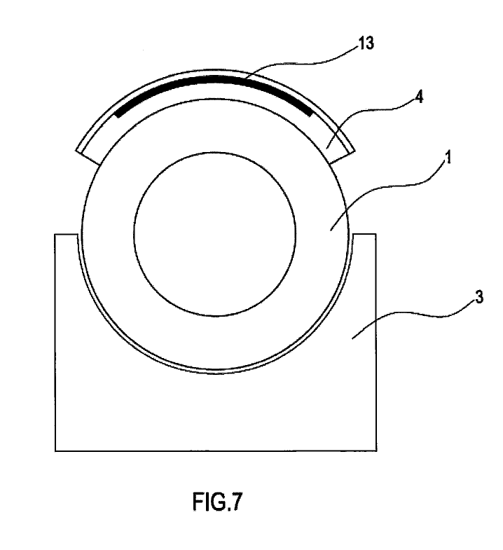

Please refer to FIGS. 6 and 7, wherein FIG. 6 shows an assembly drawing of one of embodiment for anastomosing vessels in accordance with the invention; and FIG. 7 shows an assembly drawing of another one embodiment for anastomosing vessels in accordance with the invention.

As above mentioned, the ring bodies 1. 2 can be connected by easy linking structure disposed thereon after aligning. Shown as FIG. 6, the two ring bodies 1, 2 have at least one thread hole/groove 11 for providing a stitch 12 to pass through and then linking the two vessels 5, 6. Shown as FIG. 7, the two ring bodies 1, 2 have a press-buckled structure 13 for fixing the two vessels 5, 6.

Besides, the material of the anastomosis device 100 can be bio insert or bio-absorbable.

Therefore, according to above structure, the design of ring bodies includes the advantages: (1) the inner surface of the ring bodies is capable of adhering on the outer surface of the vessels by uniform pressure; (2) the two ring bodies connected two vessels tending to be linked are easily to axial alignment, such as tenon structure; and (3) the two ring bodies have easy linking structure, such as thread hole/grooves or press-buckled structure.

In conclusion, the main difference between the invention and prior art is without turn the vessels inside out by new adhering method when operating and without damaging the vessels (wall), such as using stitch or stapler. And the anastomosis device provides the design of vessel outside support to avoid vessel collapse so as to improve vessel patency. Furthermore, by the design of easy linking structure disposed on the ring bodies, the invention can achieve the purposes of accurate vessel aligning, operating easily and precisely, and reducing the operation time.

Although the invention has been explained in relation to its preferred embodiment, it is not used to limit the invention. It is to be understood that many other possible modifications and variations can be made by those skilled in the art without departing from the spirit and scope of the invention as hereinafter claimed.

What is claimed is:

1. An anastomosis device, comprising:
a guiding base;
two ring bodies, rotatably and movably disposed relatively on a guiding base for connecting two vessels; and
a guiding member, leading the two vessels to pass through the two ring bodies respectively.

2. An anastomosis device as claimed in claim 1, wherein the two ring bodies have a tenon structure for aligning and embedding the two ring bodies.

3. An anastomosis device as claimed in claim 1, wherein an inner surface of each vessel is pressured uniformly in axial symmetry to make the outer surface of each vessel adhere and fix on an inner surface of each ring body.

4. An anastomosis device as claimed in claim 3, wherein the outer surface of each vessel is adhered and fixed on the inner surface of each ring body by a way selected from the group consisted of chemical bonding, physical absorption and mechanical retention.

5. An anastomosis device as claimed in claim 4, wherein the way of physical adsorption is performed by coating a bio-gel.

6. An anastomosis device as claimed in claim 4, wherein the way of mechanical retention is performed by distributing fine needles.

7. An anastomosis device as claimed in claim 1, wherein the two ring bodies have at least one thread hole/groove for providing a stitch to pass through and then linking the two vessels.

8. An anastomosis device as claimed in claim 1, wherein the two ring bodies have a press-buckled structure for fixing the two vessels.

9. An anastomosis device as claimed in claim 1, wherein the length of each ring body is between 0.5-10 mm and the inner diameter of each ring body is between 0.5-10 mm.

10. An anastomosis device as claimed in claim 1, wherein the radius of curvature of a bearing of the guiding base is the same as an outer diameter of each ring body.

11. An anastomosis device as claimed in claim 1, wherein the length of the guiding base is longer than the sum of the lengths of the two ring bodies.
